(19) United States
(12) Patent Application Publication
(10) Pub. No.: US 2024/0100104 A1
(43) Pub. Date: Mar. 28, 2024

(54) METHODS AND COMPOSITION FOR THE TREATMENT OF AUTISM SPECTRUM DISORDER

(71) Applicant: The General Hospital Corporation, Boston, MA (US)

(72) Inventor: Xue-Jun Kong, Lexington, MA (US)

(21) Appl. No.: 18/270,611

(22) PCT Filed: Dec. 30, 2021
(86) PCT No.: PCT/US2021/065685

§ 371 Date: Jun. 30, 2023

Related U.S. Application Data
(60) Provisional application No. 63/132,081, filed on Dec. 30, 2020.

(51) Int. Cl. A61K 35/747; A61K 38/095; A61P 25/00

(52) U.S. Cl. CPC A61K 35/747; A61K 38/095; A61P 25/00; A61K 2035/115

(57) ABSTRACT

Disclosed herein are methods and compositions useful for the treatment of Autism Spectrum Disorder (ASD). The methods and compositions include combination therapy with probiotics and oxytocin (OXT), resulting in a therapeutic synergy that exerts beneficial effects on ASD symptoms. The methods and compositions provide improvements in several measurable parameters including but not limited to: measured clinical index for ASD core symptoms, gut microbiome profile, and levels of OXT and inflammatory markers in the blood.

### CROSS-REFERENCE TO RELATED APPLICATIONS

This application claims the benefit of U.S. Provisional Application No. 63/132,081, filed Dec. 30, 2020, which is incorporated herein by reference in its entirety.

### SEQUENCE LISTING

A Sequence Listing accompanies this application and is submitted as an ASCII text file of the sequence listing named “125141_03719_ST25.txt” which is 1037 bytes in size and was created on Dec. 29, 2021. The sequence listing is electronically submitted via EFS-Web with the application and is incorporated herein by reference in its entirety.

### FIELD

The present technology relates to the field of autism spectrum disorder and related conditions, symptoms, disorders, or diseases, and compositions and methods for the treatment thereof.

### BACKGROUND

Autism spectrum disorder (ASD) is a complex neurodevelopmental disorder featuring impaired social communication and stereotypical repetitive behavioral patterns. ASD has become a serious health issue due to its rapidly rising prevalence. According to a recent report from the CDC, the prevalence of ASD has risen to 1 in 54 children.¹ However, its etiology remains elusive, and effective treatment is still largely unavailable.

Gut microbiome composition and inflammation have been reported to be involved in the pathogenesis of ASD through the gut-brain axis.² Recent evidence demonstrates that alterations in the gut microbiota of ASD individuals changes both gastrointestinal (GI) physiology and behaviors via the gut-microbiome-brain axis.^{3,4} Probiotic varieties used in both animal studies and clinical trials have demonstrated efficacy in improving ASD core symptoms.^{5,6} Evidence that probiotics have the potential to improve neuropsychiatric symptoms via the gut-brain axis is not limited to ASD. In fact, there is an entire subgroup of probiotics, known as psychobiotics, that may provide health benefits in patients with psychiatric illness.⁷ Animal studies have shown some psychobiotic strains can improve depression-like behavior,⁸ anxiety-like behavior,⁹ cognition,¹⁰ and autism-like behaviors, such as communication defect and stereotypic behaviors.¹¹ One psychobiotic, Lactobacillus Plantarum PS128 (PS128), showed ameliorative effects on depression- and anxiety-like behaviors in different mouse models.^{12,13} When administered to children with ASD, PS128 was shown to improve anxiety, rule-breaking behaviors, and hyperactivity/impulsivity.⁵

Oxytocin (OXT), a neuropeptide produced by the hypothalamus, is well-known for its ability to modulate emotional and social communication, bonding, and reward-related behaviors.¹⁴ OXT signaling is implicated in the etiology of ASD as previous studies using OXT receptor knockout mouse models exhibit autistic-like behavior, such as deficits in social interaction.¹⁵ Subsequent studies have shown that OXT treatment enhanced sociability in two mouse models of ASD.¹⁶ OXT shows promising therapeutic potential for ASD core symptoms because it can be easily administered and can work as a cost-effective treatment with minimal adverse effects. Furthermore, OXT plays an important role in the gut-brain axis and is likely inducible by certain probiotics such as Lactobacillus reuteri.¹⁴ However, potential biological connections between Lactobacillus Plantarum, including PS128, and endogenous OXT have not been studied. Moreover, the interactions between these two promising interventions, OXT, and PS128 have not been tested.

### SUMMARY

Disclosed herein are methods and compositions useful for the treatment of Autism Spectrum Disorder (ASD). The methods and compositions include combination therapy with probiotics and oxytocin (OXT), resulting in a therapeutic synergy that exerts beneficial effects on ASD symptoms. The methods and compositions provide improvements in several measurable parameters including but not limited to: measured clinical index for ASD core symptoms, gut microbiome profile, and levels of OXT and inflammatory markers in the blood.

Disclosed herein is a method of treating a subject diagnosed with or at risk of Autism Spectrum Disorder (ASD). In some embodiments, the method comprises: administering a combination therapy comprising an effective amount of (1) a probiotic composition comprising Lactobacillus plantarum, and (2) oxytocin. In some embodiments, the subject shows a statistical improvement in Clinical Global Impression (CGI) score after or during a course of treatment as compared to before treatment, or as compared to an untreated control subject. In some embodiments, the subject shows an improvement in caregiver-rated Social Responsiveness Scale 2nd edition (SRS-2) after or during a course of treatment as compared to before treatment, or as compared to an untreated control subject. In some embodiments, the subject shows an improvement in caregiver-rated Aberrant Behavior Checklist (ABC) after or during a course of treatment as compared to before treatment, or as compared to an untreated control subject.

In some embodiments of the method, the probiotic composition comprises about 1×10³ to about 1×10²⁰ CFU, 1×10⁴ to about 1×10¹⁵ CFU, 1×10⁵ to about 1×10¹² CFU, 1×10⁶ to about 1×10¹¹ CFU, or about 1×10¹⁰ to about 1×10¹¹ CFU, or about 1×10¹⁰ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 10×10¹⁰ CFU, of Lactobacillus plantarum.

In some embodiments of the method, administration comprises multiple doses of the probiotic composition over the course of about 1 week, about 2 weeks, about 3 weeks, about 4 weeks, about 5 weeks, about 2 months, about 3 months, about 4 months, about 6 months, about 1 year, or as frequently as symptoms dictate. In some embodiments, the probiotic composition is administered once per day. In some embodiments, the probiotic compositions is administered twice per day. In some embodiments the probiotic composition is administered multiple times per day. In some embodiments, the probiotic is administered orally.

In some embodiments, of the method, the probiotic composition comprises additional probiotic microorganisms.

In some embodiments of the method, administration comprises multiple doses of oxytocin over the course of about 1 week, about 2 weeks, about 3 weeks, about 4 weeks, about 5 weeks, about 2 months, about 3 months, about 4 months, about 6 months, about 1 year, or as frequently as symptoms dictate. In some embodiments, oxytocin is administered once per day. In some embodiments, oxytocin is administered twice per day. In some embodiments, oxytocin is administered multiple times per day. In some embodiments, oxytocin is administered nasally. In some embodiments, oxytocin is administered orally. In some embodiments, oxytocin is administered nasally at about 1-3 IU per day, about 2-4 IU per day, about 3-5 IU per day, about 4-6 IU per day, about 5-7 IU per day, about 6-8 IU per day, about 8-10 IU per day, about 10-20 IU per day, about 20-30 IU per day, about 30-40 IU per day, about 40-50 IU per day, about 50-100 IU per day. In some embodiments, the dosage of oxytocin is gradually increased throughout a course of administration.

In any of the disclosed methods, the oxytocin and the probiotic composition exhibit a synergistic effect.

In some embodiments of the disclosed method, the oxytocin and the probiotic composition are each administered daily. In some embodiments, the combination therapy is administered for at least about 1 week, about 2 weeks, about 3 weeks, about 4 weeks, about 5 weeks, about 2 months, about 3 months, about 4 months, about 6 months, about 1 year, or as frequently as symptoms dictate, and wherein the probiotic and the oxytocin are administered at the same time, at different times, for the same duration, or for different durations.

In any of the disclosed methods, the microbiome composition of the subject is different after the treatment as compared to before the treatment. In some embodiments, the difference comprises an increase in one or more of Eubacterium hallii, Rikenelaceae, Alistipes, Christensenellaceae, Lachnospiraceae, Blautia, Barnesiella and Ruminococcaceae.

In some embodiments of the disclosed methods, the subject shows improvement in one or more of SRS communication, SRS mannerisms, SRS motivation, SRS cognition, ABC inappropriate speech, ABC stereotypical behavior, ABC irritability, and ABC hyperactivity/non-compliance.

Disclosed herein are compositions for the treatment of ASD or like conditions, or ASD-like symptoms. In some embodiments, the composition comprises Lactobacillus plantarum, and oxytocin formulated for oral administration. In some embodiments, the compositions comprises additional probiotic microorganisms and/or additional active agents.

Disclosed herein are methods of treating a subject exhibiting one or more behavioral, psychological, or physical characteristics of Autism Spectrum Disorder (ASD), the method comprising: administering a combination therapy comprising an effective amount of (1) a probiotic composition comprising Lactobacillus plantarum, and (2) oxytocin. In some embodiments, the one or more behavioral, psychological, or physical characteristics are improved in the subject after treatment as compared to before treatment, or compared to an untreated control subject. In some embodiments, the subject shows improvement in one or more of the following: GCI scores, SRS-2 score, and ABC score.

### BRIEF DESCRIPTION OF THE DRAWINGS

FIG. 1. Flowchart of overall study design and conduct.

FIG. 2. Proportion of subjects displaying improvement in CGI-I overtime among all subjects within a treatment condition. The z-test for equality of proportions is applied and the number of subjects displaying at least minimal improvement (CGI-I≤3) in the control condition and the Probiotic+OXT condition is significantly different (P<0.05), while the changes of probiotic and OXT alone groups are non-significant, though a trend of improvement seen in both intervention groups.

FIG. 3A-C. SparCC network associations between genus level gut microbiota between subjects receiving placebo and those receiving active probiotic overtime using a SparCC cutoff of 0.7. Placebo group V1 is baseline, V2 is after placebo, V3 is after placebo added OXT; Probiotics group V1 is baseline, V2 is after probiotics, V3 is after probiotics added OXT; (A) SparCC co-occurrence network. Articulation points are marked as halos around the node. Hub score is indicated by the size of the node. (B) The number of lines or edges is significantly enriched in both OXT alone and combination groups at visit 3 (Pearson's χ²-test with Yates continuity correction, P<0.005). (C) The number of articulation points is only significantly increased in combination group at V3 compared to baseline number of articulation points (Pearson's χ²-test with Yates continuity correction, P<0.05).

FIG. 4. Heatmap of mean change in predicted functional profile based on gut microbiota abundance across four study groups. Shown changes in functional profiling indices demonstrated more changes in combination group although these changes are not significantly different when compared to the control group (P>0.05).

FIG. 5A-E. Summary of longitudinal serum marker changes and associated correlations. Absolute changes in serum OXT (A), S100B (B) and IL-1β (C) levels, comparing each treatment group against controls. Baseline serum S100B is positively correlated with ABC irritability T (D) and ABC hyperactivity/noncompliance T scores (E).

FIG. 6. Waterfall data of CGI score reduction in each group.

FIG. 7A-E. α and β diversity of fecal samples showed no significant changes in this study.

FIG. 8. Table showing summary of subject demographics and clinical indices at baseline. *Continuous data was evaluated for P-values via the Wilcoxon rank-sum test while categorical data was evaluated for intergroup differences via the Pearson's χ²-test with Yates' continuity correction.

FIG. 9. Table showing summary of improvement in socio-behavioral measures. Data are presented as mean change±SD. *Provided P-values are based on Wilcoxon rank-sum test between the mean improvement in score in the control group and the respective treatment groups.

FIG. 10. Table showing summary of identified key hub taxa based on SparCC network analysis. “+” marks that a taxon of interest (hub score>0.8) is found within the top 10 hub taxa at the given study visit and experimental group, “−” marks not found. The provided value is the hub score (the cut off score is 0.8).

FIG. 11. Table showing Spearman correlations between the microbiota relative abundance and socio-behavioral parameters before treatment for all subjects. *All presented correlations are significant at FDR<0.1.

FIG. 12. Table showing significant correlations between primary outcomes and microbiota relative abundance in probiotic group and placebo group subjects based on Spearman's rank correlation. “*” indicates FDR<0.1 based on screening of results.

### DETAILED DESCRIPTION

In this study, the inventors evaluated two promising interventions, probiotics and oxytocin, alone and in combination, against placebo control. All the interventions were well tolerated, and no major adverse events were observed. Only in the combination treatment group, we observed a trend of improvement in social and behavioral measurements (ABC and SRS), particularly in the ABC total score (P=0.077), ABC stereotyped behavior sub-score (P=0.069), and SRS-cognition sub-score (P=0.059). Meanwhile, a significant improvement of CGI was found only in the combination treatment group compared to the placebo, not in the probiotics or OXT treatment alone groups. A significant number of favorable gut microbiome network hubs (P<0.05) were also identified after combination therapy. The favorable social cognition response of combination regimen is highly correlated with the abundance of Eubacterium hallii group.

The inventors found that combination therapy elicited significant clinical improvement has not been reported previously. Previously, PS128 was found to increase dopamine and serotonin in different animal studies' 12,13; however, its relationship with oxytocin has not been tested. There is growing evidence for crucial interactions among the dopaminergic system, oxytocin/vasopressin, and serotoninergic systems in different areas of the brain that greatly influence human social behavior.^{18,19} We believe that this finding not only opens a new avenue for ASD treatment but also furthers our knowledge about the gut-brain axis and ASD pathogenesis and warrants further studies.

### Definitions

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

Wherever embodiments are described with the language “comprising,” otherwise analogous embodiments described in terms of “consisting of” and/or “consisting essentially of” are included.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value).

Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The present invention has been described in terms of one or more preferred embodiments, and it should be appreciated that many equivalents, alternatives, variations, and modifications, aside from those expressly stated, are possible and within the scope of the invention.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For purposes of this disclosure, “treating” or “treatment” describes the management and care of a patient for the purpose of combating the disease, condition, or disorder. The terms embrace both preventative, i.e., prophylactic, and palliative treatment. “Treating” includes the administration of a composition of present disclosure to prevent the onset of the symptoms or complications, alleviating the symptoms or complications, or eliminating the disease, condition, or disorder. The term “treat” and words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of this disclosure can provide any amount of any level of treatment or prevention of disease in a mammal. Furthermore, the treatment or prevention provided by the inventive method can include treatment or prevention of one or more conditions or symptoms of the disease or disease state, e.g., ASD, being treated or prevented. Also, for purposes herein, “prevention” can encompass delaying the onset of the disease, or a symptom or condition thereof for purposes of the present disclosure, “treating” or “treatment” comprises the management and care of a subject for the purpose of combating a disease, condition, or disorder. Treating includes the administration of a probiotic and oxytocin as described herein to prevent the onset of the symptoms or complications, and/or to alleviate the symptoms or complications of a disease, condition, or disorder.

As used herein, the terms “effective amount” and “therapeutically effective amount” refer to the quantity of active therapeutic agent or agents sufficient to yield a desired therapeutic response without undue adverse side effects such as toxicity, irritation, or allergic response. The specific “effective amount” will, obviously, vary with such factors as the particular condition being treated, the physical condition of the subject, the duration of the treatment, the nature of concurrent therapy (if any), and the specific formulations employed and the structure of the therapeutic or its derivatives. The exact dosage is chosen by the individual physician in view of the patient to be treated. Dosage and administration are adjusted to provide sufficient levels of the active agent(s) or to maintain the desired effect.

By “subject” or “individual” or “animal” or “patient” is meant any subject, particularly a mammalian subject, for whom diagnosis, prognosis, or therapy is desired. The present invention is generally applied to humans, but one may use the present invention for veterinary purposes. For example, one may wish to treat, or test a treatment, on commercially important farm animals, such as cows, horses, pigs, rabbits, goats, and sheep, or relevant laboratory animals, such as rats, mice, rabbits, and so on. One may also wish to treat companion animals, such as cats and dogs.

In some embodiments, the optimum effective amounts can be readily determined by one of ordinary skill in the art using routine experimentation. In some embodiments, a therapeutically effective amount is achieved by administering multiple therapeutically effective doses, e.g., over the course of a day, several days, a week, several weeks, months, or years. In some embodiments, an effective amount is administered as symptoms dictate.

Any appropriate method can be practiced to determine, detect, or monitor a subject's response to treatment according to a method provided herein. As used herein, “determining a subject's response to treatment” refers to the assessment of the results of a therapy in a subject in response to administration of a composition provided herein or to treatment according to a method provided herein.

As used herein, a Clinical Global Impression (CGI) is a scale used to measure symptom severity and treatment response. In some embodiments, the CGI is a three-item observer-rated scale that is used by clinicians and researchers to track symptom changes, e.g., prior to versus after initiating a treatment. The three items that it assesses are: 1) Severity of Illness (CGI-S), 2) Global Improvement (CGI-I), and 3) Efficacy Index (CGI-E), which is a measure of treatment effect and side effects specific to drugs that were administered. The CGI was developed for use in NIMH-sponsored clinical trials to provide a brief, stand-alone assessment of the clinician's view of the patient's global functioning prior to and after initiating a study medication. The CGI comprises two companion one-item measures evaluating the following: (a) severity of psychopathology from 1 to 7 (CGI-S) and (b) change from the initiation of treatment on a similar seven-point scale (CGI-I).

Advantages and Applications

As shown in the Examples below, the inventors observed improved ABC and SRS-2 scores and a significant improvement in CGI-Improvement (P<0.05) in the probiotics and oxytocin combination group compared to the placebo group. A significant number of favorable gut microbiome network hubs (P<0.05) were also identified after combination therapy. The favorable social cognition response of combination regimen is highly correlated with the abundance of Eubacterium hallii group.

In the Examples, the inventors evaluated and compared two promising interventions, probiotics and oxytocin, alone and in combination, against placebo control. All the interventions were well tolerated, and no major adverse events were observed. Only in the combination treatment group, did the inventors observe a trend of improvement in social and behavioral measurements (ABC and SRS), particularly in the ABC total score (P=0.077), ABC stereotyped behavior sub-score (P=0.069), and SRS-cognition sub-score (P=0.059). Meanwhile, a significant improvement of CGI was found only in the combination treatment group compared to the placebo, not in the probiotics or OXT treatment alone groups.

That the combination therapy as disclosed herein elicited significant clinical improvement has not been reported previously. Previously, PS128 was found to increase dopamine and serotonin in different animal studies^{12,13}; however, its relationship with oxytocin has not been tested. There is growing evidence for crucial interactions among the dopaminergic system, oxytocin/vasopressin, and serotoninergic systems in different areas of the brain that greatly influence human social behavior.^{18,19} This technology not only opens a new avenue for ASD treatment but also furthers our knowledge about the gut-brain axis and ASD pathogenesis.

Therapeutic Compositions

The compositions disclosed herein may include pharmaceutical (therapeutic) compositions comprising (1) a probiotic composition comprising Lactobacillus plantarum, (2) oxytocin, and (3) optionally, additional probiotic microorganisms and/or additional active agents, and may be formulated for administration to a subject in need thereof. In some embodiments, the probiotic composition and the oxytocin are provided together, e.g., in the same formulation. In some embodiments, the probiotic composition and the oxytocin are provided separately, e.g., in different formulations.

Compositions of the present disclosure can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, preservatives, and surfactants, as known in the art. The compositions also may include buffering agents (e.g., in order to maintain the pH of the composition).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”).

In some embodiments, compositions are formulated for systemic delivery, such as oral, nasal, or parenteral delivery (e.g., intraarterially, intravenously, intraperitoneally, subcutaneously, or intramuscularly).

According to some embodiments, oral administration is in the form of hard or soft gelatin capsules, pills, capsules, tablets, including coated tablets, dragees, elixirs, suspensions, liquids, gels, slurries, or syrups and controlled release forms thereof. In some embodiments, one or more of the compositions of the present disclosure are added to a food or a beverage for oral administration.

Exemplary carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrrolidone, agar, or alginic acid or a salt thereof, such as sodium alginate, may be added. Capsules and cartridges of, for example, gelatin for use in a dispenser may be formulated containing a powder mix of the compound and a suitable powder base, such as lactose or starch.

Solid dosage forms for oral administration include without limitation capsules, tablets, pills, powders, and granules. In such solid dosage forms, the active compound is admixed with at least one inert pharmaceutically acceptable carrier such as sucrose, lactose, or starch. Such dosage forms can also comprise, as it normal practice, additional substances other than inert diluents, e.g., lubricating, agents. In the case of capsules, tablets and pills, the dosage forms may also comprise buffering agents. Tablets and pills can additionally be prepared with enteric coatings. The term “enteric coating”, as used herein, refers to a coating which controls the location of composition absorption within the digestive system. Non-limiting examples for materials used for enteric coating are fatty acids, waxes, plant fibers or plastics.

Liquid dosage forms for oral administration may further contain adjuvants, such as wetting agents, emulsifying and suspending agents, and sweetening, flavoring and perfuming agents.

In some embodiments, the probiotic composition comprises about 1×10³ to about 1×10²⁰ CFU, 1×10⁴ to about 1×10¹⁵ CFU, 1×10⁵ to about 1×10¹² CFU, 1×10⁶ to about 1×10¹¹ CFU, or about 1×10¹⁰ to about 1×10¹¹ CFU, or about 1×10¹⁰ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 10×10¹⁰ CFU, of Lactobacillus plantarum.

In some embodiments, oxytocin is administered nasally at about 1-3 IU per day, about 2-4 IU per day, about 3-5 IU per day, about 4-6 IU per day, about 5-7 IU per day, about 6-8 IU per day, about 8-10 IU per day, about 10-20 IU per day, about 20-30 IU per day, about 30-40 IU per day, about 40-50 IU per day, about 50-100 IU per day

In some embodiments, the probiotic composition and the oxytocin are co-administered, i.e., are administered at the same time (e.g., if they are in the same formulation, such as an oral tablet or capsule) or at essentially the same time (e.g., if probiotic is orally administered, and the oxytocin is nasally administered). In some embodiments, the probiotic composition and the oxytocin are administered at different times.

In some embodiments, the probiotic composition is administered orally, as multiple doses over the course of about 1 week, about 2 weeks, about 3 weeks, about 4 weeks, about 5 weeks, about 2 months, about 3 months, about 4 months, about 6 months, about 1 year, or as frequently as symptoms dictate. By way of example, but not by way of limitation, in some embodiments, the probiotic composition is administered 1, 2, 3, 4, 5, or more times per day.

In some embodiments, the oxytocin is administered nasally or orally, as multiple doses over the course of about 1 week, about 2 weeks, about 3 weeks, about 4 weeks, about 5 weeks, about 2 months, about 3 months, about 4 months, about 6 months, about 1 year, or as frequently as symptoms dictate. By way of example, but not by way of limitation, in some embodiments, the oxytocin composition is administered 1, 2, 3, 4, 5, or more times per day.

### Examples

The present examples are not intended to limit the scope of the invention.

### Example 1. Effects of Combination Therapy of Probiotics and Oxytocin Among Patients with Autism Spectrum Disorder: A Randomized, Double Blinded, Placebo-Controlled Trial

Brief Summary of Background: Autism spectrum disorder (ASD) is a rapidly growing neurodevelopmental disorder. Both probiotics and oxytocin were reported to have therapeutic potential; however, the combination therapy has not yet been studied.

Brief Summary of Methods: We conducted a randomized, double-blinded, placebo-controlled, 2-stage pilot trial in 35 individuals with ASD aged 3-20 years (median=10.30 years). Subjects were randomly assigned to receive daily Lactobacillus Plantarum PS128 probiotics (6×10¹⁰ CFUs) or a placebo for 28 weeks; starting on week 16, both groups received oxytocin. The primary outcome measures tested socio-behaviors using the Social Responsiveness Scale, 2nd edition (SRS-2) and Aberrant Behavior Checklist (ABC). Measured secondary outcomes include the Clinical Global Impression (CGI) scale, gut microbiome composition, and blood inflammatory markers, including Interleukin-1β, S100 calcium-binding protein B, glial fibrillary acidic protein, myelin basic protein and oxytocin level. The outcomes were compared between the two groups at baseline, 16-weeks, and 28-weeks into treatment.

Brief Summary of Results: Overall, we observed improved ABC and SRS-2 scores and a significant improvement in CGI-Improvement (P<0.05) in the probiotics and oxytocin combination group compared to the placebo group. A significant number of favorable gut microbiome network hubs (P<0.05) were also identified after combination therapy. The favorable social cognition response of combination regimen is highly correlated with the abundance of Eubacterium hallii group.

Conclusions: Our findings indicate synergic effects between probiotics PS128 and oxytocin in ASD patients.

Results

Demographics

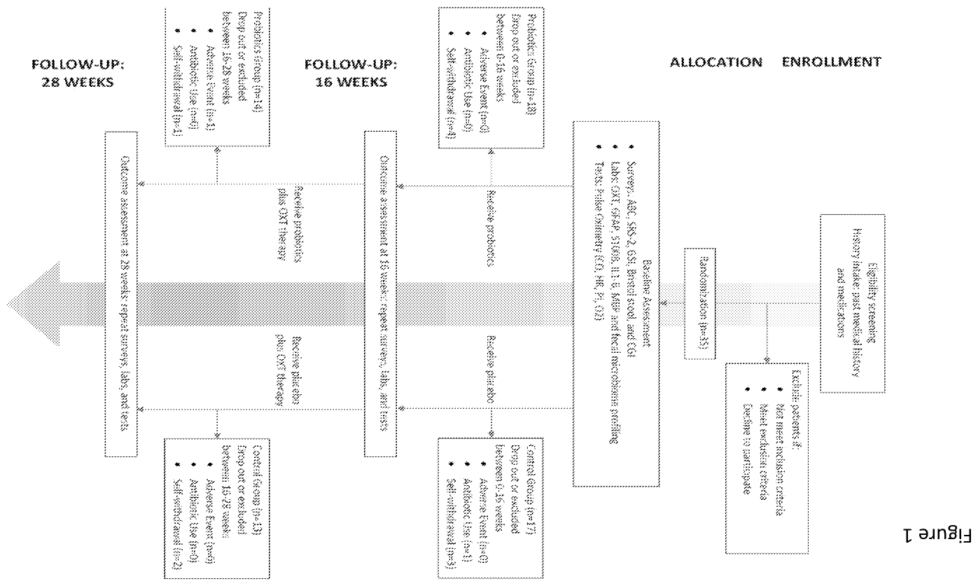

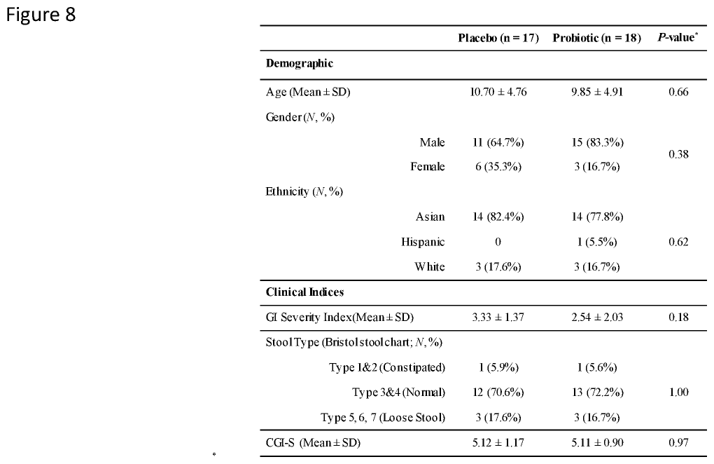

The flowchart of the study is shown in FIG. 1. Between Dec. 12, 2018 and Jun. 17, 2019, we enrolled and randomized 35 patients with ASD aged 3-20 years (median=10.30 years). The placebo group subjects had an age range of 4.69-19.70 years, while the probiotic group subjects had an age range of 3.60-18.50 years. The baseline demographic features and clinical indices of the 35 participants are shown in FIG. 8. There was no significant difference between two groups in these demographic and clinical indices (P>0.05). No serious or severe adverse events were observed. One subject was terminated due to minor nose bleeding in stage 2 that resolved quickly on its own; this subject had a history of recurrent nose bleeding related to his seasonal rhinitis. Another subject was terminated due to oral ambulatory antibiotics use for a mild upper respiratory infection. Other self-withdrawals were due to moving, travel, or other administrative reasons which were found to have no relation to the study or any adverse events. There was no significant difference of dropouts found between the two groups (P>0.05).

Socio-Behavioral Parameters and Other Clinical Indices

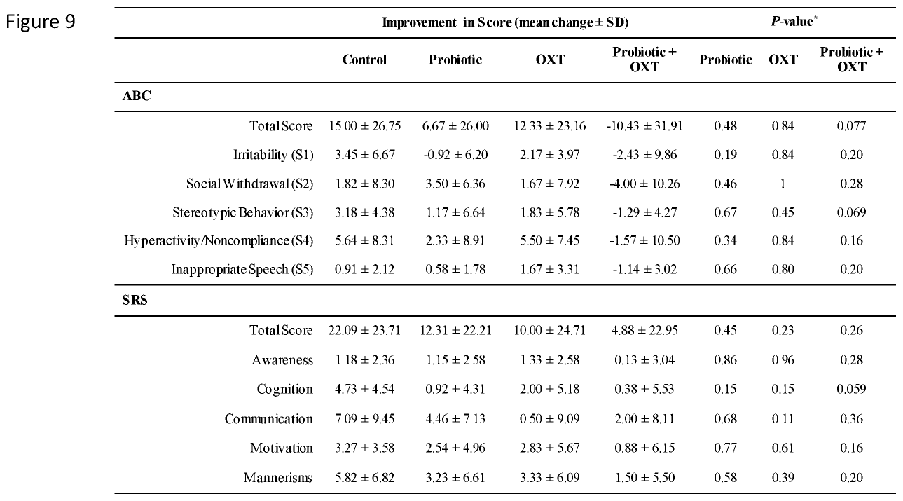

Changes in socio-behavioral parameters as measured by Aberrant Behavior Checklist (ABC) and Social Responsiveness Scale 2nd edition (SRS-2) from visit 1 to visit 2 (V2-V1) for the control group and probiotics group, and from visit 1 to visit 3 (V3-V1) for the OXT group and probiotic+OXT combination group (FIG. 9). We performed independent Wilcoxon rank-sum tests for subjects in each treatment group against the control group subjects. Trends of improvement in the total ABC score (P=0.077), stereotypic behavior score (P=0.069), and SRS-cognition score (P=0.059) were observed in combination therapy group (Probiotic+OXT), although no significant differences were observed in the total scores or subscales of the ABC and SRS (Wilcoxon rank-sum test, P>0.05).

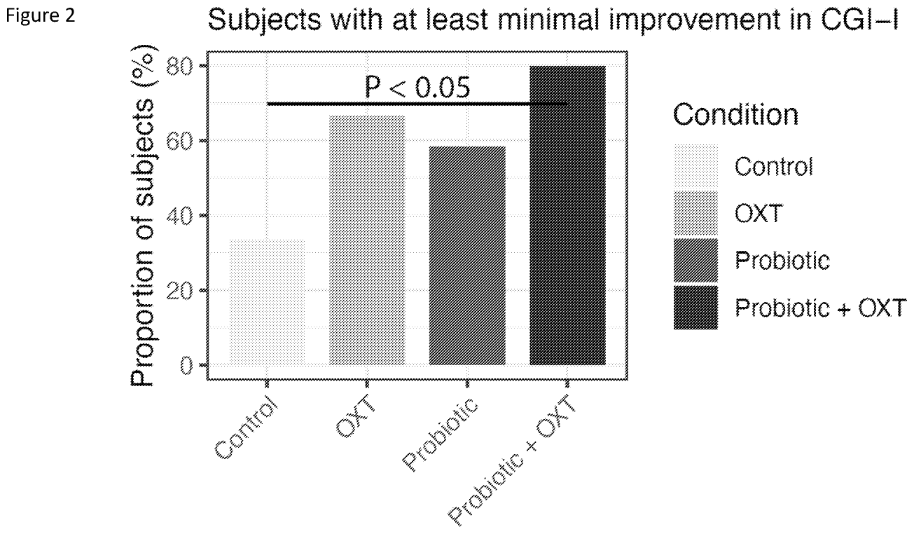

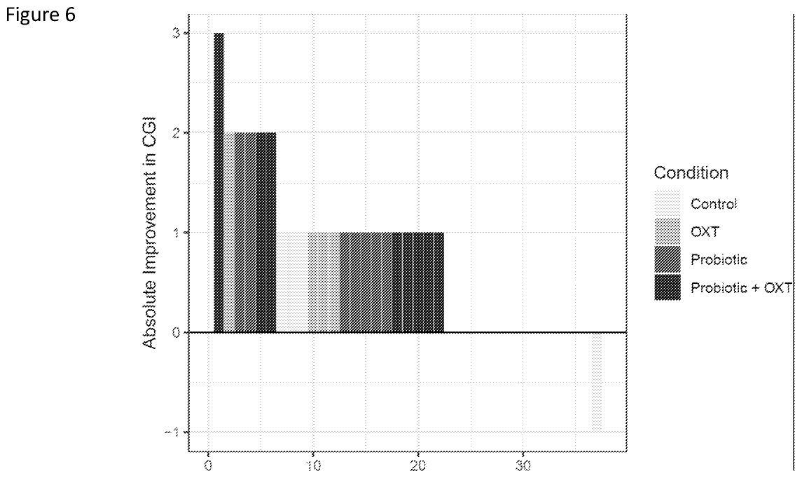

Clinical Global Impression (CGI) was assessed to evaluate ASD symptoms and the relative extent of improvement in symptoms at each visiting time. As seen in FIG. 2, the proportion of subjects showing improvement is significantly increased only in the Probiotic+OXT combination group when compared against that of the control group (Pearson's χ²-test, P<0.05), while the changes in the probiotics or OXT alone groups were non-significant, though a trend of improvement was observed in both intervention groups. The waterfall data of CGI score reduction in each group is shown in supplement FIG. 6.

GI Severity Index (GIS) showed no significant changes in three treatment groups compared with the placebo group over the treatment course (P>0.05).

Gut Microbiome

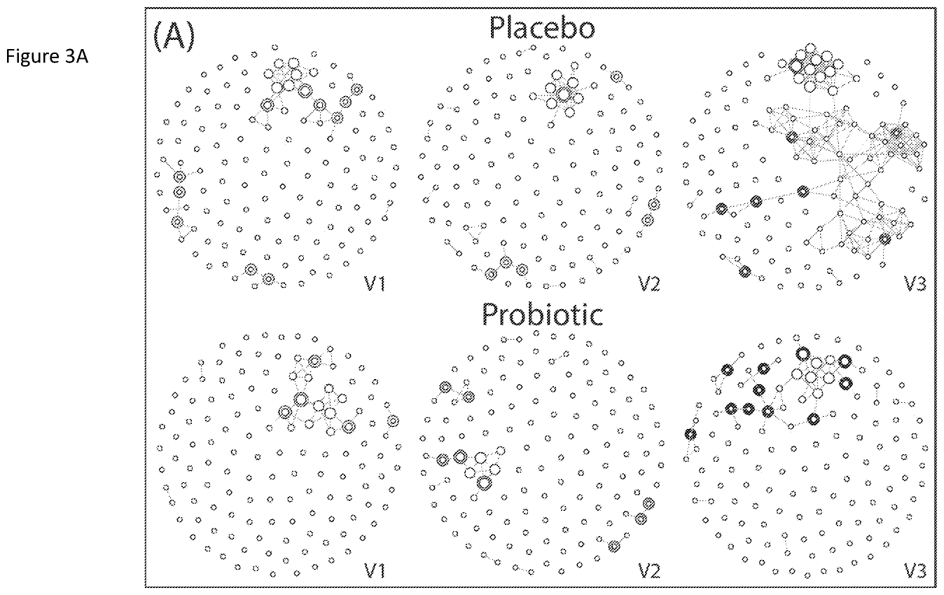

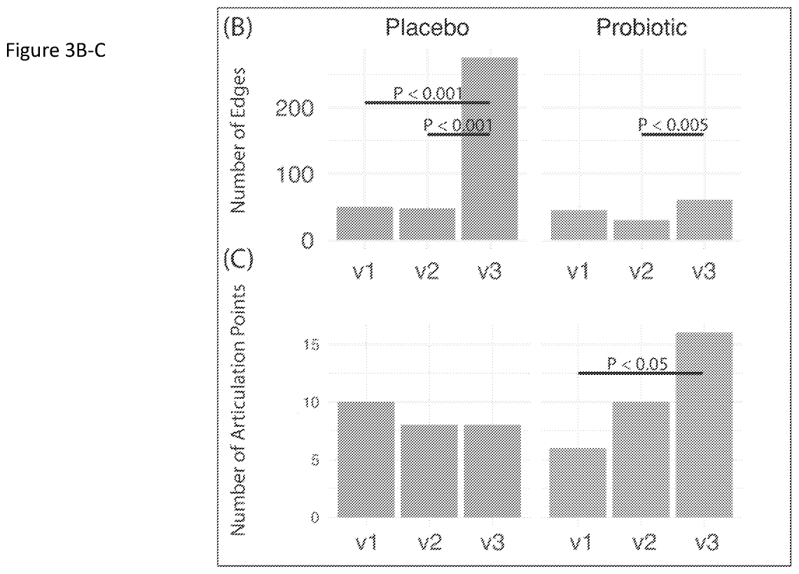

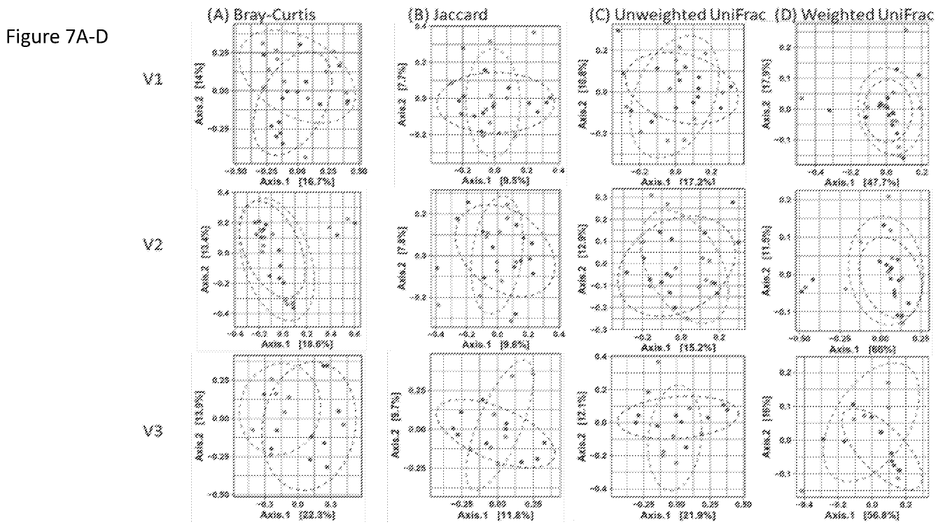

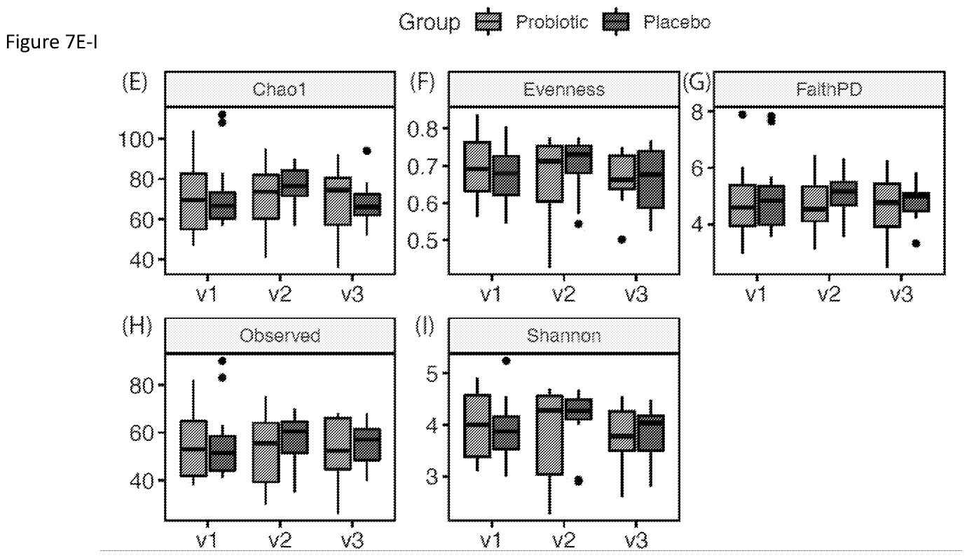

The gut microbiome was investigated by sequencing the fecal DNA. Although α and β diversity showed no significant changes in this study (FIG. 7), we found a significant increase in microbiota hubs and numbers of connection edges uniquely at V3 as compared to the two previous visits V1 and V2 (FIG. 3A) using a SparCC cutoff of 0.7. The lines or edges of the connections were significantly increased in both OXT alone group (P<0.001) and the combination group (P<0.005) (FIG. 3B), however, the number of articulation points (those with halos around the node also called “hubs”) were only significantly more in the combination group (Pearson's χ²-test with Yates continuity correction, P<0.05, FIG. 3C).

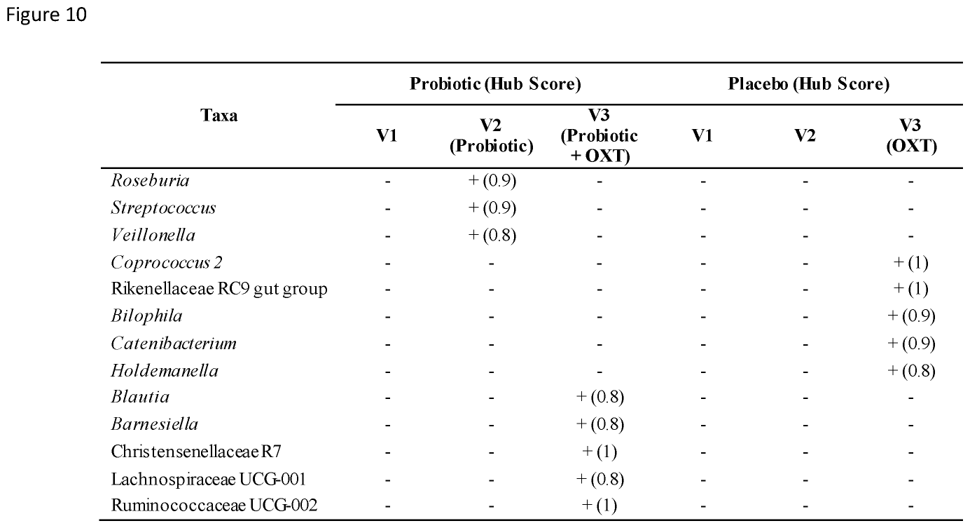

When we investigated those key hub taxa with a hub score greater than 0.8; interestingly, we found a distinct panel of hubs (marked as “+”) in the three treatment groups without overlaps. Christensenellaceae R7, Ruminococcaceae UCG-002, Lachnospiraceae UCG-001, Blautia, and Barnesiella were only present in combination therapy group; distinct hubs, Coprococcus 2, Rikenellaceae RC9, Bilophila, Catenibacterium, and Holdemanella, were only found in the OXT alone group; while Roseburia, Veillonella, and Streptococcus were only present in the probiotics group. None of the key hubs were only found in the placebo group (FIG. 10).

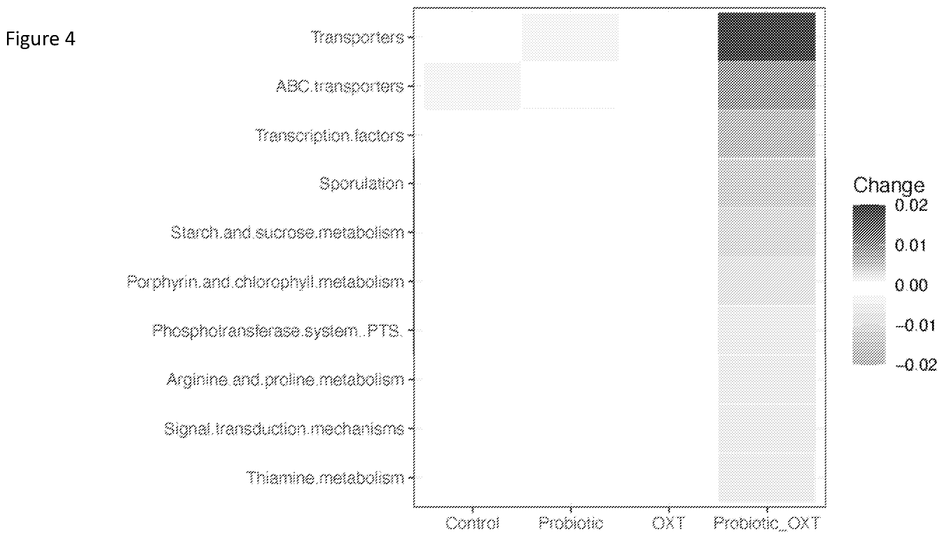

Functional gene predictive analysis indicated that several genes trended towards greater abundances in the combination group over 28-week treatment period. Notably, genes encoding transporters, ABC transporters, transcription factors, sporulation, starch and sucrose metabolism, porphyrin and chlorophyll metabolism, signal transcription metabolism, arginine and proline metabolism, and thiamine metabolism were found to be more enriched in combination groups than other groups, although the difference was not statistically significant (P>0.05, FIG. 4).

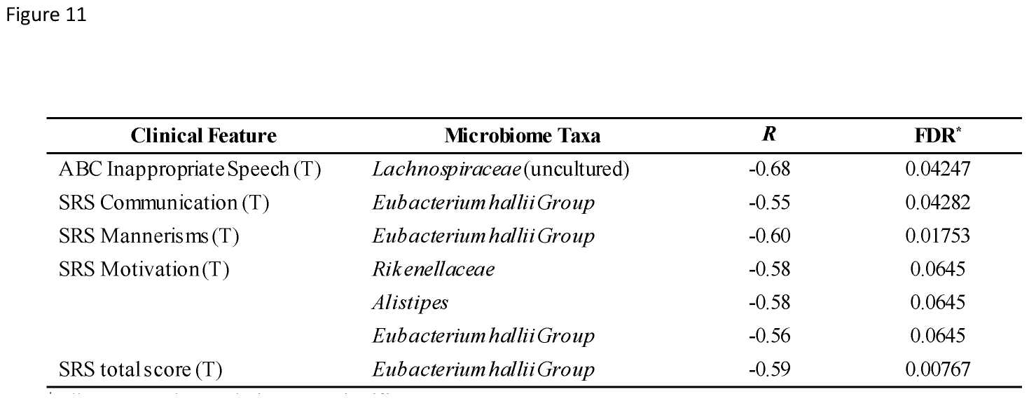

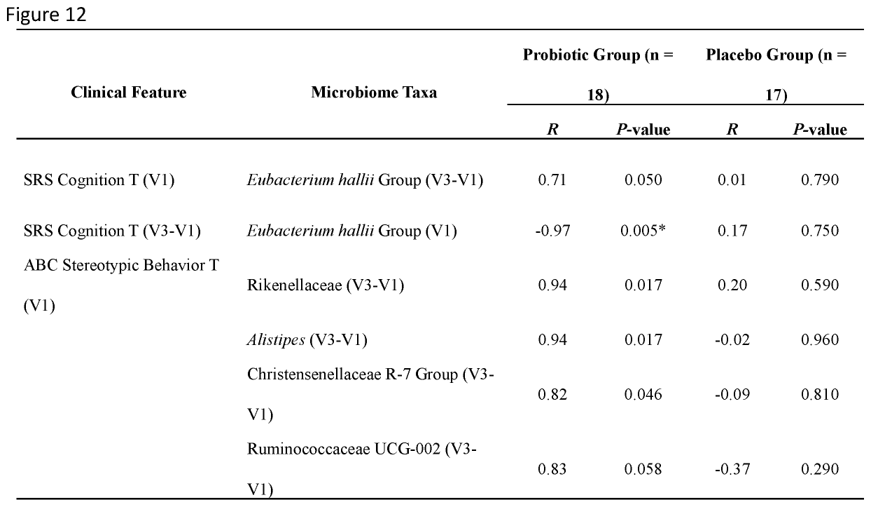

We then performed Spearman correlational analysis to assess the correlation between socio-behavioral parameters measured by ABC and SRS and microbiota relative abundance at baseline and over the course of treatment. Interestingly, the taxa Eubacterium hallii group was found to be significantly associated with total scores (R=−0.59, False Discovery Rate-adjusted P (FDR)=0.00767) and three subscales of the SRS before treatment (SRS communication: R=−0.55, FDR=0.04282); SRS mannerism: R=−0.6, FDR=0.01753; SRS motivation: R=−0.56, FDR=0.0645; FIG. 11); the strongest negative correlation was found between Eubacterium hallii group and SRS cognition score (Spearman's rho=−0.97, P=0.0048, FDR<0.1), furthermore, the absolute change (V3-V1) in Eubacterium hallii group abundance in the combination therapy group is positively correlated with baseline SRS-cognition score (Spearman's rho=0.71, P=0.05), meantime, the absolute change (V3-V1) in Rikenelaceae, Alistipes, Christensenellaceae R7, and Ruminococcaceae UCG-002 in the combination therapy group positively correlated with ABC stereotypic behavior score at baseline (FIG. 12). Of note, Rikenelaceae and Alistipes were found to be significantly correlated with SRS-motivation at baseline (FIG. 11) while Christensenellaceae R7 and Ruminococcaceae UCG-002 are two out of five important and unique hubs found only in combination treatment group (FIG. 10). Additionally, Lachnospiraceae (uncultured) was found to be negatively correlated with ABC inappropriate speech at baseline (R=−0.68, FDR=0.04247).

Blood Serum Markers

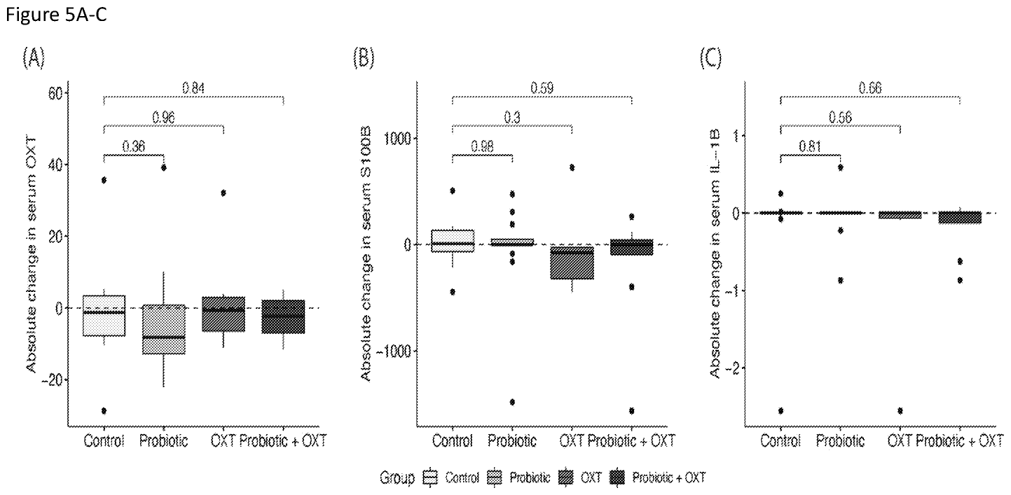

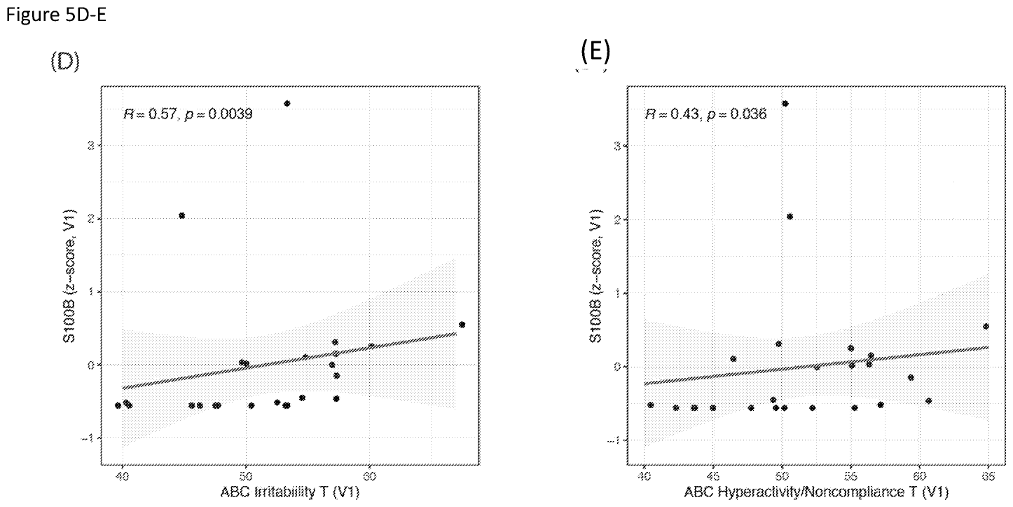

For the OXT level as measured, there was no significant changes of four groups (p>0.05) (FIG. 5A). For the inflammatory markers tested in this study, a trend of greater decrease in S100 in OXT alone group (FIG. 5B) and IL-1β levels in the combination therapy group (FIG. 5C) were observed; however, these differences were not statistically significant (Wilcoxon rank-sum test, P>0.05). By Spearman correlational analysis, we also found S100 level positively correlate with ABC irritability (FIG. 5D) and ABC hyperactivity/non-compliance score (FIG. 5E) at baseline.

Discussion

In this study, we explored and compared two promising interventions, probiotics and oxytocin, alone and in combination, against placebo control. All the interventions were well tolerated, and no major adverse events were observed. Only in the combination treatment group, we observed a trend of improvement in social and behavioral measurements (ABC and SRS), particularly in the ABC total score (P=0.077), ABC stereotyped behavior sub-score (P=0.069), and SRS-cognition sub-score (P=0.059). Meanwhile, a significant improvement of CGI was found only in the combination treatment group compared to the placebo, not in the probiotics or OXT treatment alone groups. CGI provides a brief, stand-alone assessment of the clinician's view of the patient's global functioning prior to and after initiating a study medication. The CGI-I represents the change from the initiation of treatment on a seven-point scale.¹⁷ In this study, the CGI-I was done by the clinician, who was totally blinded in treatment status and was also well acquainted with the subjects. Our finding that combination therapy elicited significant clinical improvement has not been reported previously. Previously, PS128 was found to increase dopamine and serotonin in different animal studies^{12,13}; however, its relationship with oxytocin has not been tested. There is growing evidence for crucial interactions among the dopaminergic system, oxytocin/vasopressin, and serotoninergic systems in different areas of the brain that greatly influence human social behavior.^{18,19} We believe that this finding not only opens a new avenue for ASD treatment but also furthers our knowledge about the gut-brain axis and ASD pathogenesis and warrants further studies.

Building on our finding of psychopathology improvement with combination therapy, we found some significant favorable changes in the gut microbiome over the intervention course. In particular, a significant increase in the SparCC co-occurrence network was found. The lines of the connections were significantly increased in both OXT alone group (P<0.001) and the combination group (P<0.005), however, the number of articulation points (hubs) were only significantly more in the combination group (P<0.05) not in OXT alone group (P>0.05), which suggests more critical and meaningful microbiome interactions involved in combination therapy. It's well know that an articulation point in a network is a node whose removal disconnects the network. This new finding favors the synergistic effects of the combination therapy. When examining the driving species of articulation points, we observed that the identified microbiota in the combination treatment group are unique not only from placebo group but also without overlaps with either the probiotics or OXT alone group. Among those with a high hub score (>0.8) in the combination therapy group, both Blautia and Barnesiella were previously reported to be reduced in the gut of ASD patients,²⁰⁻²² and both can promote butyrate production, which benefits gut health.²³ Christensenellaceae R7, Ruminococcaceae UCG 002, and Lachnospiraceae UCG 001 have not yet been reported in ASD, but their health benefits related to weight, gut health, and diabetes have been reported. The enrichment of these hubs overall favor improving metabolism and inflammation.²⁴⁻²⁸ The findings obtained from OXT alone group with high score hubs of Bilophila, Coprococcus-2, Holdemanella, Rikenellaceae and Catenibacterium also favor antiinflammation and gut health in general.^{5,29-34} Similarly, Roseburia, Veillonella, and Streptococcus, which were found to have high hub scores in the probiotics group, also promote anti-inflammation, gut health and additionally carbohydrate metabolism.^{5,29,35,36} In the combination group, significantly increased numbers of the articulation points are likely contributing to their better treatment responses than each treatment alone, the distinct hub panel from the single therapy groups also support the synergistic effects as observed in combination therapy group. The advance of network theory helps to disentangle the higher-order interactions that occur within microbiomes,³⁷ which could be more important than the microbiome diversity representing.

Additionally, predicted functional gene analysis suggested that several important pathways are more activated in combination group than placebo and other treatment groups. The pathways with the highest discriminative power in combination group were “Transporters” followed by “ABC transporters,” transcription factors, starch and sucrose metabolism, arginine and proline metabolism, and thiamine metabolism. ABC transporters couple energy metabolism and mediate the uptake of nutrients and physiological functions, which was found to be repressed in an ASD model with impairment of the neuronal network.^{38,39} The starch and sucrose metabolism pathways have been found to be down-regulated in ASD.⁴⁰ The human gut microbiome is a critical component of digestion, as it facilitates the breakdown of complex carbohydrates and proteins.⁴¹ Arginine has been shown to be substantially reduced in cases of gut inflammation and infection.⁴² As a metabolic precursor for nitric oxide (NO), it regulates neuron survival, differentiation, synaptic activity, and plasticity.⁴³ Thiamine (vitamin B₁) is an essential cofactor that when deficient contributes to symptoms like confusion, reduced memory, and sleep disturbances' and when adequately concentrated promotes homeostasis of a healthy gut ecosystem. These favorable findings further supported using combination therapy as a promising treatment approach than using them alone and a synergistic effect involved to facilitate energy metabolism and normal physiological functions.

Importantly, gut microbiome was found to be highly correlated with social behavioral parameters. Eubacterium hallii was found to be significantly negatively correlated with the SRS total score and sub-scores, particularly the SRS-cognition sub-score. The more enriched Eubacterium hallii correlate the lower SRS scores representing a better social function level, this strong correlation is not only observed at baseline but also with the absolute increase in the combination group at visit 3. As mentioned earlier the improvement of SRS-cognition sub-scale in combination group as showed in FIG. 9 (P=0.059) is one of the most prominent trend of improvement as observed. The higher Eubacterium hallii at baseline, the more favorable improvement of social cognition over the course of combination treatment. The lower level of social cognition (with a higher score) at baseline, the more increase of Eubacterium hallii in combination group was observed. Eubacterium hallii can utilize glucose and the fermentation intermediates acetate and lactate to form butyrate, which benefits gut health⁴⁵, however, this promising taxa has not yet been reported in ASD patients. Additionally, Christensenellaceae R7 and Ruminococcaceae UCG-002, two of the five unique hubs that were only observed in the combination treatment group (V3-V1), was found to be positively correlated with the ABC stereotypic behavior sub-score, which describes one of the ASD core symptoms. This correlation analysis further demonstrates the strong association of gut microbiome with ASD core symptoms at baseline and after a favorable treatment response in the combination group.

To further our knowledge of these treatment responses, we also measured serum oxytocin and inflammatory markers over the course of treatment. In this study, we did not find significant changes of oxytocin level in the three treatment groups when compared with placebo group.

The aberrant OXT serum levels have been reported in ASD individuals to varying degrees, sometimes decreased,^{46,47} sometimes no difference,^{48,49} and sometimes enriched compared to non-ASD controls.⁵⁰ These differences could be related to subsets of the ASD population with reduced biosynthesis or release of OXT,^{51,52} dysfunctional OXT processing dysfunction, or oxytocin receptor abnormalities.⁵³ Further studies are warranted to investigate these potential ASD subtypes and resolve these variable results, and treatment response in different sub sets.

Inflammatory mechanisms linked with ASD have been widely reported. Inflammatory cytokines were found to be significantly elevated in ASD individuals compared with healthy controls.^{54,55} Similarly, brain injury and inflammatory markers, GFAP, MBP, and S100B, have been found to be significantly enriched in ASD children than controls⁵⁵⁻⁵⁹, these brain injury markers and cytokine release subsequently trigger glial cell activation and inflammatory process in the brain.⁶⁰ In this study, we tested these four serum inflammatory markers and found that a trend of decrease of S100 in OXT group, and the decrease of IL-1β to be more pronounced in combination treatment. S100B was shown to have a significant positive correlation with the severity of problem behaviors (ABC irritability and hyperactivity scores at baseline; P<0.05).

There are several limitations of the study that deserve consideration. 1) Despite our adoption of proper recruitment and retention strategies, the participant enrollment and retention for this trial were challenging. A relatively small sample size in this pilot trial limited the statistical power and further subgroup analysis. 2) Although there was no statistical difference in clinical indices between the probiotics and placebo groups at baseline, the wide age range used in this study resulted in high subject population heterogeneity and potentially variable treatment efficacy. Future studies with a larger sample size and subgroup stratification are warranted. 3) Due to considerable Asian and other minority patients with some cultural and language barriers, in addition to multiple influencing factors on behavioral variabilities, the parent rating of social behavioral scales may be somewhat biased; 4) Sequential comparisons were not made at the same time point for the four intervention groups. The two-stage design seems inferior to simply having 4 groups with 2×2 factorial design; in this design, the prolonged treatment course might be influenced by other randomly occurring factors.

Materials and Methods

Trial Design

This clinical trial is a randomized, double-blind, and placebo-controlled study in accordance with the Consolidated Standards of Reporting Trials (CONSORT) guidelines. Subjects were randomized to two groups with 1:1 ratio into this two-stage study. To achieve a statistical power of 80% for primary outcomes with a large effect size of 0.8 (Cohen's d) assumed, a total of 60 participants (30 in each arm) were required. However, as we are primarily interested in studying the preliminary effects of the proposed treatment, we enrolled and randomized 35 subjects who were included in the data analysis. In stage 1, the probiotics group received oral probiotics PS128 while the placebo group received oral placebo for 16 weeks. In stage 2, both groups continued their respective administration and simultaneously added intranasal oxytocin spray. The treatment proceeded for a total of 28 weeks with 3 visits for outcomes measurement at 0, 16 weeks, and 28 weeks (V1, V2, and V3, respectively). While we originally planned to conduct the study outcome measurements at weeks 0, 12, and 24, we decided to prolong the study treatment for stage 1 for an additional 4 weeks based on some preliminary results. Such a change was justified by our determination that prolonged treatment of probiotic supplementation with the current strain of interest has not been previously investigated.⁵

Ethical approval of this study was issued by the Internal Review Board (IRB) of Massachusetts General Hospital (2017P001667). The trial was registered (NCT03337035), and the protocol was published previously. 6 This study was conducted according to the guidelines described in the Declaration of Helsinki. Written informed consent was obtained from competent adult subjects, or from the parents or legal guardians of children and adults with cognitive impairment according to the IRB requirements.

Compliance and safety assessments of potential adverse effects were assessed on a monthly basis via telephone check-in and self-report via Internet Research Electronic Data Capture (REDCap, v9.5.23) software. All adverse events were reported to the Human Research Committee of Massachusetts General Hospital promptly in accordance with guidelines. The Data and Safety Monitoring Plan (DSMP) was in place and approved by IRB to ensure the safety of participants, the validity of data, and the appropriate termination of this study.

Participants

Study participants were recruited through advertising posters/flyers in local communities and through ASD parent networks and workshops. Participants were included if they were 3-25 years old, have a pre-existing diagnosis of ASD confirmed by the Diagnostic and Statistical Manual of Mental Disorder (DSM-IV TR/−5) criteria, Autism Diagnostic Observation Schedule, Second Edition (ADOS-2) and/or The Autism Diagnostic Interview-Revised (ADI-R). Other inclusion criteria are: participants must have stable medications for at least 4 weeks, have no planned changes in medications or psychosocial interventions during the trial period, are willing to provide stool samples and blood in the timely manner, and are willing to participate in interviews and study procedures. A potential participant was excluded if the subject was pregnant (before or during the study), had comorbidity of other neurological and/or psychiatric disorders, such as bipolar disorders or history of a substance use disorder, was on psychotropic medications, had an active cardiovascular disease that is not controlled by medication, or had received oxytocin or probiotics treatment within the last 4 weeks. The participants were interviewed and tested in the private room of clinical research setting of Athinoula A. Martinos Center at Massachusetts General Hospital.

Randomization and Blinding

Randomization and allocation concealment were performed by a statistician who was not part of the research team, in collaboration with Massachusetts General Hospital Research Pharmacy. Randomization sampling numbers were electronically generated, and central randomization at the research pharmacy using coded drug containers that are identical in appearance were prepared by the pharmacy to ensure allocation concealment. Blinding was maintained by making the capsules look identical. Both participants and the research staff who collected the outcome data were blinded to treatment status.

Interventions

Lactobacillus Plantarum PS128 (PS128), which was isolated from a traditional Taiwan fermented mustard food,⁶¹ was deposited under DSMZ Accession No. DSM 28632. The genome architecture of PS128 was illustrated.⁶² Both animal and human studies with PS128 demonstrated great safety.^{5,12,13,63} The probiotic capsule contained only one bacteria strain (Lactobacillus Plantarum PS128). Dosage in the study was 2 capsules a day (6×10¹⁰ CFUs). Microcrystalline cellulose capsules were used as a placebo for PS128. Both probiotics and placebo capsules were free gifts obtained from Bened Biomedical Co., Ltd.

In this study, oxytocin was administered nasally. The Syntocinon® Spray (Novartis Pharma AG) is currently the most commonly used standardized oxytocin nasal spray for clinical trials worldwide. We instructed the patient and family members about the use of this spray. Dosing began with 1 puff of 4 IU daily for the first week of the second stage. Subsequently, the dosage was increased to 1 puff per nostril daily (8 IU/d) for the second week and 1 puff per nostril twice a day (16 IU/d) for the third week. The dosage was then titrated up to the maximum dose of 32 IU daily, which is 2 puffs per nostril twice a day, starting on the fourth week. The dosage of 32 IU per day has been approved as safe and adequate in even younger patients (age 3-8 years old) by a previous publication.⁶⁴ Another study reported 4-week intranasal OXT treatment (24 IU, twice daily with total 48 IU per day, which is more than the max dose in this study 32 IU per day) in 32 children with ASD, aged 6-12 years old. 65 We achieved an active IND from the FDA, and the IND number is 138827 for Syntocinon® (Pitocin, Oxytocin).

Outcomes

Primary Outcome Measures

We evaluated two primary outcome measures:

Change in caregiver-rated Social Responsiveness Scale 2nd edition (SRS-2), 66 and Change in caregiver-rated Aberrant Behavior Checklist (ABC).⁶⁷

The SRS-2 is used to assess social interest and interaction based on five subscales. We interviewed all subjects older than 4 years old. The ABC is an informant rating instrument that was empirically derived by principal component analysis. It contains 58 items that resolve onto five subscales. We interviewed all the subjects older than 5 years old.

Secondary Outcome Measures

Blood sample collection and circulating biomarker analysis. Participants presented to the Massachusetts General Hospital affiliated Athinoula A. Martinos Center for the visit after an 8 hour fast three times (week 0, week 16, and week 28). Blood was drawn and processed to obtain serum, labelled with a unique code, and stored at −80° C. Circulating OXT, MBP, GFAP, S100B, and IL-1β were measured by ELISA (R&D Systems Inc., Minneapolis, MN, USA), following manufacture's instruction.

GI symptom severity assessments. GI symptoms were assessed by the validated GI severity index (GSI), including constipation, diarrhea, stool consistency, stool smell, flatulence, abdominal pain, unexpected daytime irritability, night-time awakening, and abdominal tenderness. The stool status was scored using the Bristol Stool Chart.

Clinical Global Impression (CGI). CGI was developed for use in clinical trials to provide a brief, stand-alone assessment of the clinician's view of the patient's global functioning changes with study medication. The CGI comprises two companion one-item measures evaluating the following: (a) severity of psychopathology from 1 to 7 (CGI-S) and (b) change from the initiation of treatment on a similar seven-point scale, Clinical Global Impression-Improvement (CGI-I).¹⁷

Stool sample processing. Stool samples were collected with an OMNIgene Gut OMR-200 Collection Kit (DNA Genotek Inc.) by the participants at home under the supervision of trained parents and stored at room temperature, before de-identification and delivery or shipment to Athinoula A. Martinos Center, where stool samples were stored at −80° C. freezer. After all the experiment samples were collected after Week 28, they were hand-delivered with dry ice packaging to a laboratory at Brigham & Woman's Hospital for DNA extraction and sequencing analysis. Microbial DNA was then extracted according to the manufacturer's instructions, and DNA samples were quantified with a NanoDrop spectrophotometer. A260/A280 ratios were also measured to confirm high-purity DNA yield. Microbial 16S rRNA V4 genomic regions from total gut DNA samples were amplified with the following primers 515F (AATGATACGGCGACCACCGAGATCTACACNNNNNNNNTATGGTAATTGTGTGCCAG CMGCCGCGGTAA, SEQ ID NO: 1) and 806R (CAAGCAGAAGACGGCATACGAGATNNNNNNNNAGTCAGTCAGCCGGACTACHVGG GTWTCTAAT, SEQ ID NO: 2). PCR products were purified and analyzed using a Bioanalyzer DNA kit, followed by quantification with real-time PCR. DNA libraries were pooled and sequenced on an Illumina MiSeq next-generation sequencing system (Illumina; San Diego, CA, USA) using a V4 2×250 bp paired-end protocol with overlapping reads.

Statistical Analysis

Data analyses were performed based on the intention-to-treat principle. The primary outcomes for the treatment comparisons were the changes in the scores of SRS and ABC (SRS T score, ABC T score). Secondary outcomes measurement includes CGI, GSI, levels of serum markers, and the gut microbiome.

An independent sample t-test/Wilcoxon rank-sum test for continuous variables was used to detect between-group differences in the measurement changes over the intervention course. Paired sample t-test and Wilcoxon signed-rank test were used to test the within-group difference in the primary outcomes and secondary outcomes before and after the intervention (V2-V1 and V3-V1). The z-test for equality of proportions without continuity correction was applied to differences in the proportion of subjects displaying change in secondary outcome measures.

We additionally performed a stratified analysis based on baseline SRS/ABC score, GI condition, and neuroinflammation/neuro-injury serum marker levels.

Sequencing data were processed and analyzed with a QIIME 2,⁶⁸ and alpha diversity was calculated by Chao-1, Faith PD, Evenness, and Observed OTUs using the Phyloseq R package. Beta diversity, weighted UniFrac, unweighted UniFrac, Bray-Curtis, and Jaccard were analyzed.

SparCC co-abundance networks were constructed to examine the longitudinal change in associations between gut microbiota. 69 Correlations with magnitudes greater than a SparCC cutoff of 0.7 were considered significant. Identified hub taxa and the respective hub scores are indicated by the size of the circle. False Discovery Rate (FDR)-based Type-1 error control was made per study visit on a group-wise basis across all assessed variables. This was done via MaAsLin2 for assessed correlations between primary and secondary outcomes and blood serum marker concentrations against microbiota relative abundances.⁷⁰ Significant correlations were considered at an FDR<0.1.

PICRUSt-1 (phylogenetic investigation of communities by reconstruction of unobserved states) is a computational approach to predict the functional composition of a metagenome using marker gene data and a database of reference genomes and was applied to the current 16S dataset. The relative change in abundance of each feature abundance (ASVs or pathways) between visits V1 and V2 (V2-V1) and visits V1 and V3 (V3-V1) were computed for each experimental subject. Then differential analyses were performed on the relative changes between probiotics and placebo group with Wilcoxon rank-sum test. All 16S rRNA raw data have been submitted to Sequence Read Archive (SRA) database of The National Center for Biotechnology Information SAMN16687792-SAMN16687860.

### REFERENCES

1. Maenner M J, Shaw K A, Baio J, EdS1, Washington A, Patrick M, DiRienzo M, Christensen D L, Wiggins L D, Pettygrove S, et al. Prevalence of Autism Spectrum Disorder Among Children Aged 8 Years—Autism and Developmental Disabilities Monitoring Network, 11 Sites, United States, 2016. MMWR Surveillance Summaries. 2020; 69(4):1. doi:10.15585/mmwr.ss6904a1
2. Alam R, Abdolmaleky H M, Zhou J-R. Microbiome, inflammation, epigenetic alterations, and mental diseases. American Journal of Medical Genetics Part B: Neuropsychiatric Genetics. 2017; 174(6):651-660. doi:10.1002/ajmg.b.32567
3. Fung T C, Olson C A, Hsiao E Y. Interactions between the microbiota, immune and nervous systems in health and disease. Nature neuroscience. 2017; 20(2):145.
4. Kong X, Liu J, Cetinbas M, Sadreyev R, Koh M, Huang H, Adeseye A, He P, Zhu J, Russell H, et al. New and Preliminary Evidence on Altered Oral and Gut Microbiota in Individuals with Autism Spectrum Disorder (ASD): Implications for ASD Diagnosis and Subtyping Based on Microbial Biomarkers. Nutrients. 2019; 11(9). doi:10.3390/nu11092128
5. Liu Y-W, Liong M T, Chung Y-CE, Huang H-Y, Peng W-S, Cheng Y-F, Lin Y-S, Wu Y-Y, Tsai Y-C. Effects of Lactobacillus plantarum PS128 on Children with Autism Spectrum Disorder in Taiwan: A Randomized, Double-Blind, Placebo-Controlled Trial. Nutrients. 2019; 11(4). doi:10.3390/nu11040820
6. Kong X-J, Liu J, Li J, Kwong K, Koh M, Sukijthamapan P, Guo J J, Sun Z J, Song Y. Probiotics and oxytocin nasal spray as neuro-social-behavioral interventions for patients with autism spectrum disorders: a pilot randomized controlled trial protocol. Pilot and Feasibility Studies. 2020; 6:20. doi:10.1186/s40814-020-0557-8
7. Dinan T G, Stanton C, Cryan J F. Psychobiotics: a novel class of psychotropic. Biological Psychiatry. 2013; 74(10):720-726. doi:10.1016/j.biopsych.2013.05.001
8. Desbonnet L, Garrett L, Clarke G, Bienenstock J, Dinan T G. The probiotic Bifidobacteria infantis: An assessment of potential antidepressant properties in the rat. Journal of Psychiatric Research. 2008; 43(2):164-174. doi:10.1016/j.jpsychires.2008.03.009
9. Savignac H M, Kiely B, Dinan T G, Cryan J F. Bifidobacteria exert strain-specific effects on stress-related behavior and physiology in BALB/c mice. Neurogastroenterology and Motility: The Official Journal of the European Gastrointestinal Motility Society. 2014; 26(11):1615-1627. doi:10.1111/nmo.12427
10. Davari S, Talaei S A, Alaei H, Salami M. Probiotics treatment improves diabetes-induced impairment of synaptic activity and cognitive function: behavioral and electrophysiological proofs for microbiome-gut-brain axis. Neuroscience. 2013; 240:287-296. doi:10.1016/j.neuroscience.2013.02.055
11. Hsiao E Y, McBride S W, Hsien S, Sharon G, Hyde E R, McCue T, Codelli J A, Chow J, Reisman S E, Petrosino J F, et al. Microbiota modulate behavioral and physiological abnormalities associated with neurodevelopmental disorders. Cell. 2013; 155(7):1451-1463. doi:10.1016/j.cell.2013.11.024
12. Liu Y-W, Liu W-H, Wu C-C, Juan Y-C, Wu Y-C, Tsai H-P, Wang S, Tsai Y-C. Psychotropic effects of Lactobacillus plantarum PS128 in early life-stressed and naïve adult mice. Brain Research. 2016; 1631:1-12. doi:10.1016/j.brainres.2015.11.018
13. Liu W-H, Chuang H-L, Huang Y-T, Wu C-C, Chou G-T, Wang S, Tsai Y-C. Alteration of behavior and monoamine levels attributable to Lactobacillus plantarum PS128 in germ-free mice. Behavioural Brain Research. 2016; 298(Pt B):202-209. doi:10.1016/j.bbr.2015.10.046
14. Erdman S E, Poutahidis T. Microbes and Oxytocin: Benefits for Host Physiology and Behavior. International Review of Neurobiology. 2016; 131:91-126. doi:10.1016/bs.irn.2016.07.004
15. Pobbe R L H, Pearson B L, Defensor E B, Bolivar V J, Young W S, Lee H-J, Blanchard D C, Blanchard R J. Oxytocin receptor knockout mice display deficits in the expression of autism-related behaviors. Hormones and Behavior. 2012; 61(3):436-444. doi:10.1016/j.yhbeh.2011.10.010
16. Teng B L, Nikolova V D, Riddick N V, Agster K L, Crowley J J, Baker L K, Koller B H, Pedersen C A, Jarstfer M B, Moy S S. Reversal of social deficits by subchronic oxytocin in two autism mouse models. Neuropharmacology. 2016; 105:61-71. doi:10.1016/j.neuropharm.2015.12.025
17. Busner J, Targum S D. The clinical global impressions scale: applying a research tool in clinical practice. Psychiatry (Edgmont (Pa.: Township)). 2007; 4(7):28-37.
18. Skuse D H, Gallagher L. Dopaminergic-neuropeptide interactions in the social brain. Trends in Cognitive Sciences. 2009; 13(1):27-35. doi:10.1016/j.tics.2008.09.007
19. Marotta R, Risoleo M C, Messina G, Parisi L, Carotenuto M, Vetri L, Roccella M. The Neurochemistry of Autism. Brain Sciences. 2020; 10(3). doi:10.3390/brainsci10030163
20. Inoue R, Sakaue Y, Sawai C, Sawai T, Ozeki M, Romero-Perez G A, Tsukahara T. A preliminary investigation on the relationship between gut microbiota and gene expressions in peripheral mononuclear cells of infants with autism spectrum disorders. Bioscience, Biotechnology, and Biochemistry. 2016; 80(12):2450-2458. doi:10.1080/09168451.2016.1222267
21. Luna R A, Oezguen N, Balderas M, Venkatachalam A, Runge J K, Versalovic J, Veenstra-VanderWeele J, Anderson G M, Savidge T, Williams K C. Distinct Microbiome-Neuroimmune Signatures Correlate With Functional Abdominal Pain in Children With Autism Spectrum Disorder. Cellular and Molecular Gastroenterology and Hepatology. 2017; 3(2):218-230. doi:10.1016/j.jcmgh.2016.11.008
22. Averina O V, Kovtun A S, Polyakova S I, Savilova A M, Rebrikov D V, Danilenko V N. The bacterial neurometabolic signature of the gut microbiota of young children with autism spectrum disorders. Journal of Medical Microbiology. 2020; 69(4):558-571. doi:10.1099/jmm.0.001178
23. Lustgarten M S. The Role of the Gut Microbiome on Skeletal Muscle Mass and Physical Function: 2019 Update. Frontiers in Physiology. 2019; 10:1435. doi:10.3389/fphys.2019.01435
24. Garcia-Mantrana I, Selma-Royo M, Alcantara C, Collado M C. Shifts on Gut Microbiota Associated to Mediterranean Diet Adherence and Specific Dietary Intakes on General Adult Population. Frontiers in Microbiology. 2018; 9:890. doi:10.3389/fmicb.2018.00890
25. Wang Y, Zhang H, Zhu L, Xu Y, Liu N, Sun X, Hu L, Huang H, Wei K, Zhu R. Dynamic Distribution of Gut Microbiota in Goats at Different Ages and Health States. Frontiers in Microbiology. 2018; 9:2509. doi:10.3389/fmicb.2018.02509
26. Guo M, Li Z. Polysaccharides isolated from Nostoc commune Vaucher inhibit colitis-associated colon tumorigenesis in mice and modulate gut microbiota. Food & Function. 2019; 10(10):6873-6881. doi:10.1039/c9fo00296k
27. Gao B, Zhong M, Shen Q, Wu Y, Cao M, Ju S, Chen L. Gut microbiota in early pregnancy among women with Hyperglycaemia vs. Normal blood glucose. BMC pregnancy and childbirth. 2020; 20(1):284. doi:10.1186/s12884-020-02961-5
28. Ma S, You Y, Huang L, Long S, Zhang J, Guo C, Zhang N, Wu X, Xiao Y, Tan H. Alterations in Gut Microbiota of Gestational Diabetes Patients During the First Trimester of Pregnancy. Frontiers in Cellular and Infection Microbiology. 2020; 10:58. doi:10.3389/fcimb.2020.00058
29. Strati F, Cavalieri D, Albanese D, De Felice C, Donati C, Hayek J, Jousson O, Leoncini S, Renzi D, Calabro A, et al. New evidences on the altered gut microbiota in autism spectrum disorders. Microbiome. 2017; 5(1):24. doi:10.1186/s40168-017-0242-1
30. Tomova A, Soltys K, Kemenyova P, Karhanek M, Babinska K. The Influence of Food Intake Specificity in Children with Autism on Gut Microbiota. International Journal of Molecular Sciences. 2020; 21(8). doi:10.3390/ijms21082797
31. Berding K, Donovan S M. Diet Can Impact Microbiota Composition in Children With Autism Spectrum Disorder. Frontiers in Neuroscience. 2018; 12:515. doi:10.3389/fnins.2018.00515
32. El Hage R, Hernandez-Sanabria E, Calatayud Arroyo M, Props R, Van de Wiele T. Propionate-Producing Consortium Restores Antibiotic-Induced Dysbiosis in a Dynamic in vitro Model of the Human Intestinal Microbial Ecosystem. Frontiers in Microbiology. 2019 [accessed 2020 October 16]; 10. https://www.frontiersin.org/articles/10.3389/fmicb.2019.01206/full. doi:10.3389/fmicb.2019.01206
33. Kang D-W, Park J G, Ilhan Z E, Wallstrom G, Labaer J, Adams J B, Krajmalnik-Brown R. Reduced incidence of Prevotella and other fermenters in intestinal microflora of autistic children. PloS One. 2013; 8(7):e68322. doi:10.1371/journal.pone.0068322
34. Jarett J K, Carlson A, Rossoni Serao M, Strickland J, Serfilippi L, Ganz H H. Diets with and without edible cricket support a similar level of diversity in the gut microbiome of dogs. Peed. 2019; 7:e7661. doi:10.7717/peerj.7661
35. Zhang M, Ma W, Zhang J, He Y, Wang J. Analysis of gut microbiota profiles and microbe-disease associations in children with autism spectrum disorders in China. Scientific Reports. 2018; 8(1):13981. doi:10.1038/s41598-018-32219-2
36. De Angelis M, Francavilla R, Piccolo M, De Giacomo A, Gobbetti M. Autism spectrum disorders and intestinal microbiota. Gut Microbes. 2015; 6(3):207-213. doi:10.1080/19490976.2015.1035855
37. Layeghifard M, Hwang D M, Guttman D S. Disentangling Interactions in the Microbiome: A Network Perspective. Trends in Microbiology. 2017; 25(3):217-228. doi:10.1016/j.tim.2016.11.008
38. Liu F, Horton-Sparks K, Hull V, Li R W, Martinez-Cerdelio V. The valproic acid rat model of autism presents with gut bacterial dysbiosis similar to that in human autism. Molecular Autism. 2018; 9:61. doi:10.1186/s13229-018-0251-3
39. Iritani S, Torii Y, Habuchi C, Sekiguchi H, Fujishiro H, Yoshida M, Go Y, Iriki A, Isoda M, Ozaki N. The neuropathological investigation of the brain in a monkey model of autism spectrum disorder with ABCA13 deletion. International Journal of Developmental Neuroscience: The Official Journal of the International Society for Developmental Neuroscience. 2018; 71:130-139. doi:10.1016/j.ijdevneu.2018.09.002
40. Rose D R, Yang H, Serena G, Sturgeon C, Ma B, Careaga M, Hughes H K, Angkustsiri K, Rose M, Hertz-Picciotto I. Differential immune responses and microbiota profiles in children with autism spectrum disorders and co-morbid gastrointestinal symptoms. Brain, behavior, and immunity. 2018; 70:354-368.
41. Oliphant K, Allen-Vercoe E. Macronutrient metabolism by the human gut microbiome: major fermentation by-products and their impact on host health. Microbiome. 2019; 7(1):91. doi:10.1186/s40168-019-0704-8
42. Fritz J H. Arginine cools the inflamed gut. Infection and Immunity. 2013; 81(10):3500-3502. doi:10.1128/IAI.00789-13
43. Tripathi M K, Kartawy M, Amal H. The role of nitric oxide in brain disorders: Autism spectrum disorder and other psychiatric, neurological, and neurodegenerative disorders. Redox Biology. 2020; 34:101567. doi:10.1016/j.redox.2020.101567
44. Dhir S, Tarasenko M, Napoli E, Giulivi C. Neurological, Psychiatric, and Biochemical Aspects of Thiamine Deficiency in Children and Adults. Frontiers in Psychiatry. 2019; 10:207. doi:10.3389/fpsyt.2019.00207
45. Udayappan S, Manneras-Holm L, Chaplin-Scott A, Belzer C, Herrema H, Dallinga-Thie G M, Duncan S H, Stroes E S G, Groen A K, Flint H J, et al. Oral treatment with Eubacterium hallii improves insulin sensitivity in db/db mice. NPJ biofilms and microbiomes. 2016; 2:16009. doi:10.1038/npjbiofilms.2016.9
46. Husarova V M, Lakatosova S, Pivovarciova A, Babinska K, Bakos J, Durdiakova J, Kubranska A, Ondrejka I, Ostatnikova D. Plasma Oxytocin in Children with Autism and Its Correlations with Behavioral Parameters in Children and Parents. Psychiatry Investigation. 2016; 13(2):174-183. doi:10.4306/pi.2016.13.2.174
47. Zhang H-F, Dai Y-C, Wu J, Jia M-X, Zhang J-S, Shou X-J, Han S-P, Zhang R, Han J-S. Plasma Oxytocin and Arginine-Vasopressin Levels in Children with Autism Spectrum Disorder in China: Associations with Symptoms. Neuroscience Bulletin. 2016; 32(5):423-432. doi:10.1007/s12264-016-0046-5
48. Miller M, Bales K L, Taylor S L, Yoon J, Hostetler C M, Carter C S, Solomon M. Oxytocin and vasopressin in children and adolescents with autism spectrum disorders: sex differences and associations with symptoms. Autism Research: Official Journal of the International Society for Autism Research. 2013; 6(2):91-102. doi:10.1002/aur.1270
49. Parker K J, Garner J P, Libove R A, Hyde S A, Hornbeak K B, Carson D S, Liao C-P, Phillips J M, Hallmayer J F, Hardan A Y. Plasma oxytocin concentrations and OXTR polymorphisms predict social impairments in children with and without autism spectrum disorder. Proceedings of the National Academy of Sciences of the United States of America. 2014; 111(33):12258-12263. doi:10.1073/pnas.1402236111
50. Jansen L M C, Gispen-de Wied C C, Wiegant V M, Westenberg H G M, Lahuis B E, van Engeland H. Autonomic and neuroendocrine responses to a psychosocial stressor in adults with autistic spectrum disorder. Journal of Autism and Developmental Disorders. 2006; 36(7):891-899. doi:10.1007/s10803-006-0124-z
51. Dadds M R, Moul C, Cauchi A, Dobson-Stone C, Hawes D J, Brennan J, Ebstein R E. Methylation of the oxytocin receptor gene and oxytocin blood levels in the development of psychopathy. Development and Psychopathology. 2014; 26(1):33-40. doi:10.1017/S0954579413000497
52. Jin D, Liu H-X, Hirai H, Torashima T, Nagai T, Lopatina O, Shnayder N A, Yamada K, Noda M, Seike T, et al. CD38 is critical for social behaviour by regulating oxytocin secretion. Nature. 2007; 446(7131):41-45. doi:10.1038/nature05526
53. Watanabe T, Otowa T, Abe O, Kuwabara H, Aoki Y, Natsubori T, Takao H, Kakiuchi C, Kondo K, Ikeda M, et al. Oxytocin receptor gene variations predict neural and behavioral response to oxytocin in autism. Social Cognitive and Affective Neuroscience. 2017; 12(3):496-506. doi:10.1093/scan/nsw150
54. Masi A, Quintana D S, Glozier N, Lloyd A R, Hickie I B, Guastella A J. Cytokine aberrations in autism spectrum disorder: a systematic review and meta-analysis. Molecular Psychiatry. 2015; 20(4):440-446. doi:10.1038/mp.2014.59
55. Guloksuz S A, Abali O, Aktas Cetin E, Bilgic Gazioglu S, Deniz G, Yildirim A, Kawikova I, Guloksuz S, Leckman J F. Elevated plasma concentrations of S100 calcium-binding protein B and tumor necrosis factor alpha in children with autism spectrum disorders. Revista Brasileira De Psiquiatria (Sao Paulo, Brazil: 1999). 2017; 39(3):195-200. doi:10.1590/1516-4446-2015-1843
56. Abou-Donia M B, Suliman H B, Siniscalco D, Antonucci N, ElKafrawy P. De novo Blood Biomarkers in Autism: Autoantibodies against Neuronal and Glial Proteins. Behavioral Sciences (Basel, Switzerland). 2019; 9(5). doi:10.3390/bs9050047
57. Esnafoglu E, Ayyildiz S N, Cirrik S, Erturk E Y, Erdil A, Dagli A, Noyan T. Evaluation of serum Neuron-specific enolase, S100B, myelin basic protein and glial fibrilliary acidic protein as brain specific proteins in children with autism spectrum disorder. International Journal of Developmental Neuroscience: The Official Journal of the International Society for Developmental Neuroscience. 2017; 61:86-91. doi:10.1016/j.ijdevneu.2017.06.011
58. Mostafa G A, Al-Ayadhi L Y. A lack of association between hyperserotonemia and the increased frequency of serum anti-myelin basic protein auto-antibodies in autistic children. Journal of Neuroinflammation. 2011; 8:71. doi:10.1186/1742-2094-8-71
59. Gonzalez-Gronow M, Cuchacovich M, Francos R, Cuchacovich S, Blanco A, Sandoval R, Gomez C F, Valenzuela J A, Ray R, Pizzo S V. Catalytic autoantibodies against myelin basic protein (MBP) isolated from serum of autistic children impair in vitro models of synaptic plasticity in rat hippocampus. Journal of Neuroimmunology. 2015; 287:1-8. doi:10.1016/j.jneuroim.2015.07.006
60. Kern J K, Geier D A, Sykes L K, Geier M R. Relevance of Neuroinflammation and Encephalitis in Autism. Frontiers in Cellular Neuroscience. 2015; 9:519. doi:10.3389/fncel.2015.00519
61. Chao S-H, Wu R-J, Watanabe K, Tsai Y-C. Diversity of lactic acid bacteria in suan-tsai and fu-tsai, traditional fermented mustard products of Taiwan. International Journal of Food Microbiology. 2009; 135(3):203-210. doi:10.1016/j.ijfoodmicro.2009.07.032
62. Liu W-H, Yang C-H, Lin C-T, Li S-W, Cheng W-S, Jiang Y-P, Wu C-C, Chang C-H, Tsai Y-C. Genome architecture of Lactobacillus plantarum PS128, a probiotic strain with potential immunomodulatory activity. Gut Pathogens. 2015; 7:22. doi:10.1186/s13099-015-0068-y
63. Liao P-L, Wu C-C, Chen T-Y, Tsai Y-C, Peng W-S, Yang D-J, Kang J-J. Toxicity Studies of Lactobacillus plantarum PS128TM Isolated from Spontaneously Fermented Mustard Greens. Foods (Basel, Switzerland). 2019; 8(12). doi:10.3390/foods8120668
64. Yatawara C J, Einfeld S L, Hickie I B, Davenport T A, Guastella A J. The effect of oxytocin nasal spray on social interaction deficits observed in young children with autism: a randomized clinical crossover trial. Molecular Psychiatry. 2016; 21(9):1225-1231. doi:10.1038/mp.2015.162
65. Parker K J, Oztan O, Libove R A, Sumiyoshi R D, Jackson L P, Karhson D S, Summers J E, Hinman K E, Motonaga K S, Phillips J M, et al. Intranasal oxytocin treatment for social deficits and biomarkers of response in children with autism. Proceedings of the National Academy of Sciences of the United States of America. 2017; 114(30):8119-8124. doi:10.1073/pnas.1705521114
66. Constantino J, Gruber C. Social responsive scale (SRS) manual. Los Angeles, CA: Western Psychological Services; 2005.
67. Aman M G, Singh N N, Stewart A W, Field C J. The aberrant behavior checklist: a behavior rating scale for the assessment of treatment effects. American Journal of Mental Deficiency. 1985; 89(5):485-491.
68. Bolyen E, Rideout J R, Dillon M R, Bokulich N A, Abnet C C, Al-Ghalith G A, Alexander H, Alm E J, Arumugam M, Asnicar F, et al. Reproducible, interactive, scalable and extensible microbiome data science using QIIME 2. Nature Biotechnology. 2019; 37(8):852-857. doi:10.1038/s41587-019-0209-9
69. Friedman J, Alm E J. Inferring correlation networks from genomic survey data. PLoS computational biology. 2012; 8(9):e1002687. doi:10.1371/journal.pcbi.1002687
70. Mallick, et al. Multivariable Association Discovery in Population-scale Meta-omics Studies. 2020; ((In Submission)).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of treating a subject diagnosed with or at risk of Autism Spectrum Disorder (ASD), the method comprising: administering a combination therapy comprising an effective amount of (1) a probiotic composition comprising Lactobacillus plantarum, and (2) oxytocin.

2. The method of claim 1, wherein the subject shows a statistical improvement in Clinical Global Impression (CGI) score after or during a course of treatment as compared to before treatment.

3. The method of claim 1, wherein the subject shows an improvement in caregiver-rated Social Responsiveness Scale 2nd edition (SRS-2) after or during a course of treatment as compared to before treatment.

4. The method of claim 1, wherein the subject shows an improvement in caregiver-rated Aberrant Behavior Checklist (ABC) after or during a course of treatment as compared to before treatment.

5. The method of claim 1, wherein the probiotic composition comprises about 1×10³ to about 1×10²⁰ CFU, 1×10⁴ to about 1×10¹⁵ CFU, 1×10⁵ to about 1×10¹² CFU, 1×10⁶ to about 1×10¹¹ CFU, or about 1×10¹⁰ to about 1×10¹¹ CFU, or about 1×10¹⁰ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 10×10¹⁰ CFU, of Lactobacillus plantarum.

6. The method of claim 1, wherein administration comprises multiple doses of the probiotic composition over the course of about 1 week, about 2 weeks, about 3 weeks, about 4 weeks, about 5 weeks, about 2 months, about 3 months, about 4 months, about 6 months, about 1 year, or as frequently as symptoms dictate.

7. The method of claim 1, wherein the probiotic composition is administered once per day, or multiple times per day.

8.-9. (canceled)

10. The method of claim 1, wherein the probiotic is administered orally.

11. The method of claim 1, wherein administration comprises multiple doses of oxytocin over the course of about 1 week, about 2 weeks, about 3 weeks, about 4 weeks, about 5 weeks, about 2 months, about 3 months, about 4 months, about 6 months, about 1 year, or as frequently as symptoms dictate.

12. The method of claim 1, wherein oxytocin is administered once per day, or multiple times per day.

13.-14. (canceled)

15. The method of claim 1, wherein the oxytocin is administered nasally or orally.

16. (canceled)

17. The method of claim 1, wherein the oxytocin is administered nasally at about 1-3 IU per day, about 2-4 IU per day, about 3-5 IU per day, about 4-6 IU per day, about 5-7 IU per day, about 6-8 IU per day, about 8-10 IU per day, about 10-20 IU per day, about 20-30 IU per day, about 30-40 IU per day, about 40-50 IU per day, about 50-100 IU per day.

18. The method of claim 1, wherein the dosage of oxytocin is gradually increased throughout a course of administration.

19. The method of claim 1, wherein the oxytocin and the probiotic composition exhibit a synergistic effect.

20.-21. (canceled)

22. The method of claim 1, wherein the microbiome composition of the subject is different after the treatment as compared to before the treatment.

23. The method of claim 22, wherein the difference comprises an increase in one or more of Eubacterium hallii, Rikenelaceae, Alistipes, Christensenellaceae, Lachnospiraceae, Blautia, Barnesiella and Ruminococcaceae.

24. (canceled)

25. A composition comprising Lactobacillus plantarum, and oxytocin formulated for oral administration.

26. The method of claim 1, wherein the probiotic composition comprises additional probiotic microorganisms and/or additional active agents.

27. (canceled)

28. A method of treating a subject exhibiting one or more behavioral, psychological, or physical characteristics of Autism Spectrum Disorder (ASD), the method comprising: administering a combination therapy comprising an effective amount of (1) a probiotic composition comprising Lactobacillus plantarum, and (2) oxytocin.

29. The method of claim 28, wherein the one or more behavioral, psychological, or physical characteristics are improved in the subject after treatment as compared to before treatment.

30. (canceled)
